(19) United States
(12) Patent Application Publication
(10) Pub. No.: US 2024/0120096 A1
(43) Pub. Date: Apr. 11, 2024

(54) Computational Method And System For Diagnostic And Therapeutic Prediction From Multimodal Data

(71) Applicant: HypaHub, Inc., San Jose, CA (US)

(72) Inventors: Bayo Lau, San Jose, CA (US); Ashley Sylvia Lee, Rockville, MD (US); Tarsus Thomas Lam, Murrieta, CA (US); Paul Merrill, Sunnyvale, CA (US); Hugo Y.K. Lam, Sunnyvale, CA (US)

(21) Appl. No.: 18/377,518

(22) Filed: Oct. 6, 2023

Related U.S. Application Data
(60) Provisional application No. 63/413,833, filed on Oct. 6, 2022.

(51) Int. Cl. G16H 50/20; G16B 40/00

(52) U.S. Cl. CPC G16H 50/20; G16B 40/00

(57) ABSTRACT

A computational method, apparatus and system for diagnostic and therapeutic prediction from multimodal data is provided for using machine learning to predict medical therapeutic methods using multimodal data. The computational method, apparatus and system for diagnostic and therapeutic prediction from multimodal data may include a biomarker and subtype identification aspect, multimodality aspect, machine learning aspect, and training aspect. A method for using machine learning to predict medical therapeutic methods, which may include targets, drugs, or combinations of drugs, using multimodal data using the computational system for diagnostic and therapeutic prediction from multimodal data is also provided.

### CROSS REFERENCE TO RELATED APPLICATIONS

This application claims the benefit of U.S. Provisional Application Ser. No. 63/413,833, filed on Oct. 6, 2022, which is hereby incorporated by reference herein.

### FIELD OF THE INVENTION

The present disclosure relates to a computational system for diagnostic and therapeutic prediction from multimodal data. More particularly, the disclosure relates to using machine learning to predict medical therapeutic methods using multimodal data.

### BACKGROUND

Many diseases can be associated with a patient's molecular profile, including their genomic, transcriptomic, proteomic, and epigenetic alterations, as well as their phenotypic profile, such as images and medical records. Some or all of these multimodal profiles can contribute to disease diagnosis. Further, targeted therapy aims to select the optimal medical treatment based on some or all of such profiles. While targeted therapy has benefits over non-targeted alternatives in many cases, the treatment response rate of many diseases is still suboptimal and requires non-trivial effort to improve. For example, most cancers have a response rate to immunotherapy of less than 20%.

Developing precision diagnosis requires the identification and interpretation of biomarkers from the patient's profiles. Targeted therapy further matches beneficial treatments, targeting the disease with patients exhibiting such biomarkers. As a point of reference, without limitation, finding drugs targeting human diseases could mean matching one or more chemical compounds out of an infinite number of possibilities to a handful of biomarkers selected from a biological system with over 3,000,000,000 genomic locations and 25,000 genes.

Machine learning (ML) is a computational method that learns patterns from training data and makes predictions for previously unseen data. ML has been successful in many disciplines such as image analysis, in which large amounts of data are available and where the nature of training data is often similar to the previously unseen data. ML applications to targeted therapy have been challenging because of limited data and batch heterogeneity. In the prior art, the data is bottlenecked by the scarcity of systematic molecular and treatment profiling of patients compared to the large number of possible biomarker-treatment combinations. Additionally, in studies and medical trials, the techniques used for recording clinical data, selecting patients, preparing biospecimens, measuring molecular profiles, and analyzing data inherently introduce undesirable data variabilities and hard-to-reproduce biases, which hamper direct cross-dataset comparison, impacting an ML model's ability to generalize from its training to new, incoming patient data.

Therefore, a need exists to solve the deficiencies present in the prior art. What is needed is a multimodal drug and treatment discovery apparatus and method that solves some or all of the problems of the prior art. What is needed is a drug and treatment discovery apparatus and method that operates a system that benefits from training machine learning models using multimodal data. What is needed is an apparatus and method that operates a system to diagnose and suggest therapeutic solutions using a predictive model. What is needed is a drug and treatment discovery apparatus and method that operates a system that can leverage training data that is distinct from a predictive application. What is needed is a drug and treatment discovery apparatus and method that operates a system that uses an inspectable machine learning model.

### SUMMARY

An aspect of the disclosure advantageously provides a multimodal drug and treatment discovery apparatus and method. An aspect of the disclosure advantageously provides and otherwise operates a drug and treatment discovery system that benefits from training machine learning models using multimodal data. An aspect of the disclosure advantageously provides a system to diagnose and suggest therapeutic solutions using a predictive model. An aspect of the disclosure advantageously provides and operates a drug and treatment discovery system that can leverage training data that is distinct from a predictive application. An aspect of the disclosure advantageously provides a drug and treatment discovery apparatus and method that operates a system that uses an inspectable machine learning model.

The present disclosure enables a machine learning computational system which identifies biomarkers and subtypes, as well as predicts medical treatments, such as drugs, and their responses based on multimodal data. The modalities can include, without limitation, genomics, transcriptomics, proteomics, radiomics, radio genomics, spatial transcriptomics, spatial proteomics, and clinical information. The training data can be either homogeneous or heterogeneous compared to the application data with which predictions are made. Further, the system can be at least partially operated on personal computers and/or distributed computing infrastructure, as well as be customized, controlled, and monitored by command-line, interactive graphical interfaces, and/or other human-computer interfaces that would be appreciated by those of skill in the art after having the benefit of this disclosure.

A preferred embodiment of this invention comprises an apparatus comprising one or more components (e.g., a personal computer or other type of computer) that operates a system and/or number of steps for computational machine learning-based normalization, harmonization, and improvement of data signals from one or more modalities of measurements. This preferred embodiment comprise at least six modules, which may be together in one or more other modules, or split into multiple modules, with different names as longs as their functions are performed. These modules comprise (a) a data input module configured to receive data from one or more biospecimens with homogeneous or heterogeneous natures, including but not limited to genomic, transcriptomic, proteomic, and epigenetic data; (b) a preprocessing module configured to execute M-space signal partition, summary and smoothing on methylation signal data; (c) a preprocessing module configured to perform transferable quantile normalization for transcriptomic signal data; (d) a preprocessing module configured to perform a reference-free DNA copy number estimation module adapted to estimate DNA copy numbers from heterogeneous sample types and measurement methods; (e) Coherence-Variance unsupervised feature selection or target-aware clustering for supervised feature summary; and (f) a machine learning module incorporating supervised and/or unsupervised learning methods to process said data, said machine learning module further configured to perform patient stratification, biomarker discovery, and prediction of drug response based on said data.

A preprocessing module of this embodiment may further comprise means to execute M-space signal partition, summary and smoothing by applying an algorithm for the enhancement of methylation signal data to achieve harmonization and data accuracy improvement.

A preprocessing module of this embodiment may execute transferable quantile normalization for transcriptomic signal data using steps to achieve harmonization and data accuracy improvement amid heterogenous and noisy biological signals.

A preprocessing module of this embodiment may execute reference-free DNA copy number estimation module of this embodiment may further employ steps to estimate DNA copy numbers from biospecimens with highly heterogeneous natures and varying measurement methods, eliminating the need for reference samples.

The Coherence-Variance unsupervised feature selection step or target-aware clustering for supervised feature summary may identify a small number of relevant features (or biomarkers) with improved signal quality to achieve harmonization and data accuracy improvement amid heterogeneous and noisy biological signals.

Another preferred embodiment of this invention comprises a method for improving the accuracy of biomarker discovery, patient stratification, and prediction of drug response, comprising the combination of 2 or more of following steps: (a) receiving data from one or more modality measurements of one or more biospecimens; (b) applying M-space signal smoothing to methylation signal data; (c) performing transferable quantile normalization for transcriptomic signal data; (d) estimating DNA copy numbers using a reference-free method for heterogeneous sample types and measurement methods; (e) executing Coherence-Variance unsupervised feature selection or target-aware clustering for supervised feature summary; and (f) utilizing supervised and/or unsupervised learning methods to process said data for biomarker discovery, patient stratification, and prediction of drug response.

Another preferred embodiment of this invention comprises a method for computational machine learning-based normalization, harmonization, and improvement of data signals from one or more modalities of measurements. This method comprises: (a) receiving data using a data input module from one or more biospecimens with homogeneous or heterogeneous natures, including but not limited to genomic, transcriptomic, proteomic, and epigenetic data; (b) executing using a preprocessing module M-space signal partition, summary and smoothing on methylation signal data; (c) transferable quantile normalization for transcriptomic signal data; (d) estimating using a reference-free DNA copy number estimation module adapted to estimate DNA copy numbers from heterogeneous sample types and measurement methods; (e) Coherence-Variance unsupervised feature selection or target-aware clustering for supervised feature summary; and (f) incorporating supervised and/or unsupervised learning methods to process said data using a machine learning module, said machine learning module further configured to perform patient stratification, biomarker discovery, and prediction of drug response based on said data.

Another preferred embodiment of this invention comprises a method of generating a therapeutic treatment response prediction, a biomarker prediction and a patient subtype prediction for a patient using at least one biospecimen from the patient This method comprises: (a) training a machine learning module with modality data (preferably from two or more modalities and more preferred from more than three modalities), the modality data comprising data from genomics, transcriptomics, proteomics, radiomics, radio genomics, spatial transcriptomics, spatial proteomics, and/or other clinical information from multiple patients, wherein the machine learning module analyzes the modality data, and wherein the analysis of the modality data comprises the identification and ranking of transcriptomic, genomic and epigenetic biomarkers of the modality data; (b) generating a model from step a. and data from the at least one biospecimen from the patient, the data from the patient comprising genomic, transcriptomic, proteomic, radiomic, radio genomic, spatial transcriptomic, spatial proteomic, and/or other clinical information; and (c) generating from the model from step b., a treatment response prediction, a biomarker prediction, and a patient subtype prediction for the patient.

Another preferred embodiment of this invention comprises an apparatus for predicting treatment responses, biomarkers, and patient subtypes concerning a therapeutic treatment for patients. This apparatus comprises (a) a machine learning module that accepts modality data (preferably from two or more modalities and more preferred from more than three modalities) from patients for analysis, the modality data comprising data from genomics, transcriptomics, proteomics, radiomics, radio genomics, spatial transcriptomics, spatial proteomics, and/or other clinical information from multiple patients, and the analysis comprises identifying and ranking transcriptomic, genomic and epigenetic biomarkers of the modality data; and (b) a model generating module that accepts the analysis from the machine learning module and data from at least one biospecimen from a patient, the data from the patient comprising genomic, transcriptomic, proteomic, radiomic, radio genomic, spatial transcriptomic, spatial proteomic, and/or other clinical information, and wherein the model generating module generates a model that predicts treatment response, biomarkers, and patient subtypes with respect to treatments for that patient.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a block diagram of an illustrative analytical workflow, according to an embodiment of this disclosure.

FIG. 2 is a diagram of a contour plot of treatment response versus an epigenetic feature extracted via standard measure, according to an embodiment of this disclosure.

FIG. 3 is a diagram of an illustrative box plot of treatment response versus a genomic copy number feature extracted by the standard measure, according to an embodiment of this disclosure.

FIG. 4 is a diagram of an illustrative visualization of subtyping based on multimodal features, according to an embodiment of this disclosure.

FIG. 5 is a diagram of an illustrative box plot of Lapatinib response predictions calculated using only mutation and RNA expression modalities, according to an embodiment of this disclosure.

FIG. 6 is a diagram of an illustrative box plot of Lapatinib response predictions calculated using mutation, RNA expression, DNA copy number variation, and DNA methylation modalities, according to an embodiment of this disclosure.

FIG. 7 is a table diagram of illustrative biomarkers along with their degree of efficacy for the predicted treatment response, according to an embodiment of this disclosure.

FIG. 8 is a diagram of an illustrative heatmap visualization of subtypes predicted for in vivo patients, according to an embodiment of this disclosure.

FIG. 9 is a block diagram of an illustrative computerized apparatus upon which a system enabled by this disclosure may operate, according to an embodiment of this disclosure.

FIG. 10 is an illustration of Coherence-Variance unsupervised feature selection using the Laplacian score (LSAuto) versus mean absolute deviation (Mad) for selected and unselected features. The large number of light-colored round dots are the unselected features, the small number of dark round dots are selected features. Contour lines are the kernel density plot of features. Features are selected based on their position relative to the density properties of the 2-dimensional space. For illustration, landmarks such as the stars and straight lines indicate an interesting region from which the features are selected.

### DETAILED DESCRIPTION

The following disclosure is provided to describe various embodiments of a computational system for diagnostic and therapeutic prediction from multimodal data. Skilled artisans will appreciate additional embodiments and uses of the present invention that extend beyond the examples of this disclosure. Terms included by any claim of a corresponding nonprovisional patent application are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term machine learning, as it is used throughout this disclosure, is defined as a computational method that learns patterns from training data and makes predictions for previously unseen data. The term in vitro, as it is used throughout this disclosure, is defined as a controllable drug and treatment discovery and testing environment outside of a living patient. The term in vivo, as it is used throughout this disclosure, is defined as being associated with a living organism in which drugs and treatments may be administered and observed.

Examples of Certain Technical Problems Solved by this Invention

This invention solves and/or more effectively addresses technical problems that others in the past have had predicting the most useful treatments for particular patients. While biomarkers were often used in discovery, diagnostic, and prognostic settings, established computation methodologies often can reap benefits from only a limited number of markers in one or a few modalities (e.g., only one from genomic, transcriptomic, epigenetic, and proteomic modalities) measured from homogeneous samples with fine-tuned, homogeneous experimental conditions and methodology. Such methodologies have profound limitations that are addressed and substantially overcome by our invention.

First, the requirement of homogeneous experimental conditions puts a severe limitation to the number of data points usable in machine learning methodologies, which require training data and test data to be similar. Because of the rapid development of modern biotechnologies, experiments done in different projects and organizations at different time can be different. For example, each project, such as a clinical trial, would measure, with its own methodology, only tens to hundreds of samples. Combining different datasets can be challenging, if possible at all, and doing so would often result in a decreased signal strength that counteracts the increased number of samples. This invention addresses the requirement of homogenous experimental conditions. The ability to process data with high heterogeneity (e.g., as heterogeneous as in vitro cell line versus in vivo patient) in certain embodiments of this invention allows machine learning to learn from a higher number of data points aggregated from heterogeneous collections of projects and thus increasing the power of the machine learning model. Further, such processing allows the trained model to make predictions on future samples (such as an in vivo patient sample) which could also be heterogeneous compared to the training set (such as in vitro cell lines as an extreme case).

Second, the limitation in combining modalities has been an on-going challenge in the field. Genomic, transcriptomic, epigenetic, and proteomic measurements quantify the many-body biological systems from different point-of-views. While the resulting data modalities do contain some aspects of the system that are coherent across the modalities, each modality can also measure information that is unique to itself. Therefore, methods such as those taught by embodiments of this invention that can reap benefits from additional modalities and construct a more holistic view of the disease state. Machine learning methods have historically struggled to gain benefits from these extra biological modalities. Here, the current invention in certain preferred embodiments can systematically reap benefits from integrating at least four modalities: DNA mutation, DNA copy number mutation, transcriptomic, and epigenetic data modalities.

By solving the aforementioned limitations, this invention provides the benefits from both an increased number of samples and an increased number of modalities, while remaining effective for the predictive tasks for future samples.

Certain embodiments of this invention solve these technical problems with quantitative and processing improvements over existing methods, in a manner that has never before been applied to these problems. These embodiments apply machine learning with quantitative improvements to all available data (“multimodal data”) that is provided from potentially many sources, and sources that have not been necessarily associated with predictive value based on previous methodologies. These embodiments also then apply predictive models with quantitative improvements to this machine learning and a particular patient's data to identify and rank or otherwise predict therapeutic success, identify patient subtypes, and identify useful biomarkers. Particular tools were also developed for this invention that have never been applied in this manner before, which includes (a) an algorithm for the enhancement of methylation signal data to execute M-space signal partitions and summary, (b) steps for identifying relevant features from the data to execute coherence-variance unsupervised selection, (c) steps to achieve harmonization and data accuracy improvement to execute transferable quantile normalization for transcriptomic signal data, and (d) DNA-copy number estimation that estimates DNA copy numbers from biospecimens with highly heterogeneous natures, using varying measurement methods, and which eliminates or at least reduces the need for reference samples. One application is to apply this invention to a particular drug to treat a particular ailment in a particular patient.

In an especially preferred embodiment of this invention, the prediction method and system will be implemented as a sophisticated computational pipeline. The pipeline will be a software that can run on users' computers, a software that runs on the cloud, or a Software as a Service (SaaS). The software consists of modules implementing the improved computational algorithms and the machine learning models. It will also integrate with other preexisting modality data such as annotation data. Users are expected to supply their multimodal data to the software and the software, after running the data through different modules, will produce the diagnostic and therapeutic predictions.

Examples of novel tools that were developed for embodiments of this invention include the following:
1. For methylation data, the chain of process that is applied in certain embodiments is a new solution, which comprises a) transforming into m-space b) grouping by, for example gene annotation, c) grouping by degree-of-correlation or by unsupervised cluster, d) applying a summary like average or Bayesian Gaussian Mixture Model. This method may be implemented to work even across in vitro cell-line samples and in vivo patient samples.
2. The Coherence-Variance methodology for unsupervised clustering for the omic data modalities that are used is a new solution. The simultaneous use of coherence and variance metrics with the data used and applied as done in certain embodiments is an example of what is a new solution to apply here. Others might uses Variance (such as MAD) to select a feature, and some might consider a “Laplacian score”, but that is not all that this invention teaches. In addition, certain embodiments of this invention automatically calculate the parameters to calculate “Laplacian score”, instead of manually tuning the parameters as is done in the past. Thus, embodiments of this invention provide a way to combine the use of Variance (for example Mad) and Coherence (for example LSAuto), which significantly lowers the amount of features needed, as well as being more concordant with biology, when compared to certain previous work which needed both RAN data and methylation data.
3. Certain embodiments of this invention use reference-free copy number normalization, which is a new solution applied here. This especially applies to heterogeneous samples like in vitro cell lines and in vivo patient samples. Reference 3 teaches that cell line copy number data is different from patient copy number and that, in general, such cell line data is highly distorted. Most copy-number analysis would require a “reference”, such as healthy cells along with tumor cells of the same individual, or a healthy cohort to “calibrate” the measurements of tumor from different individuals. Further, this “reference” is in general not available for cell-lines because they are by definition a lineage of cells. The method of this invention in these embodiments is a novel and effective in a way that, after its application, one can train a model with cell-line data, and the model can be applied to patient data, with no need of a “reference” sample. Thus, the method in these embodiments is effective in dealing with this extreme sample heterogeneity and it provides for the of building a training model with low-cost and numerous cell line samples and then applying that to high-cost and rare patient samples.
4. Certain embodiments of this invention provide reference-free transferable quantile normalization, which is a new solution applied here. While quantile normalization is available, it requires multiple samples to normalize one sample. The problem comes in with how to normalize if there is only one patient to test after training the model, with, for example 1,000 previous patients. Previous approaches used “rank normalization”, but that approach loses the numerical scale. In the embodiment of this invention using reference-free transferable quantile normalization, the transform vector is computed from the training data, and then applied to the in-coming new patient, even if there is only one new patient, and the numerical scale is maintained.
5. Certain embodiments of this invention combine four or more modalities, which works effectively only after applying some or all of the four steps discussed above in this section, which improve the predictive power of the model. Certain of the previously reported attempts at solutions did not perform these steps and even concluded that some of the modalities were not useful, often concluding, for example, that copy number and/or methylation information is not useful. Certain embodiments of this invention show that by preparing the signal carefully with these novel approaches, and then integrating them, the predictive model is quantitatively improved.

### Further Description of Preferred Embodiments

Various aspects of the present disclosure will now be described in detail, without limitation. Certain preferred embodiments are described above in the Summary. In the following disclosure, a computational system for diagnostic and therapeutic prediction from multimodal data will be discussed. Those of skill in the art will appreciate alternative labeling of the computational system for diagnostic and therapeutic prediction from multimodal data as a multimodal treatment discovery system, machine learning assistance treatment prediction system, drug and treatment discovery system using multimodal approach, the invention, or other similar names. Similarly, those of skill in the art will appreciate alternative labeling of the computational system for diagnostic and therapeutic prediction from multimodal data as a multimodal drug and treatment discovery method, predictive treatment method using multimodal training data, machine learning operation for treatment discovery using multiple sources for training, method, operation, the invention, or other similar names. Skilled readers should not view the inclusion of any alternative labels as limiting in any way. Additionally, while some of the examples provided throughout this disclosure are discussed in the context of cancer research, those of skill in the art will not view such examples as limiting and will appreciate the systems and methods discussed through this disclosure may also apply to additional diagnostic, treatment, therapeutic, drug discovery, and other related applications.

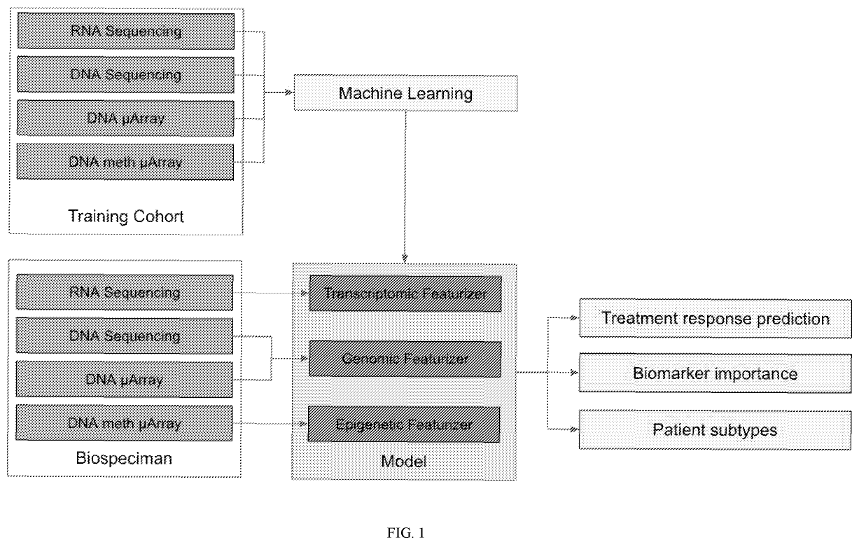

Referring now to FIGS. 1-9, the computational system for diagnostic and therapeutic prediction from multimodal data will now be discussed in more detail. The computational system for diagnostic and therapeutic prediction from multimodal data may include a biomarker and subtype identification aspect, multimodality aspect, machine learning aspect, training aspect, and additional components that will be discussed in greater detail below. The computational system for diagnostic and therapeutic prediction from multimodal data may operate one or more of these components interactively with other components for using machine learning to predict medical therapeutic methods using multimodal data. The predictive output may focus on variability between patients and medical conditions, for example, considering subtypes of patients, predictions for what types of drugs and/or treatments will work for patients, comparison diagnostics, full scale diagnostics, and other indications of variability. Considering these indications, the predictive output may advantageously be patient-targeted, considerably improving over the generalized recommendations known in the drug treatment discovery techniques of the prior art.

According to an embodiment enabled by this disclosure, a machine learning computational system is discussed to identify biomarkers and subtypes, predict medical treatments such as drugs, and predict responses based on multimodal data. Modalities may include, without limitation, genomics, transcriptomics, proteomics, radiomics, radio genomics, spatial transcriptomics, spatial proteomics, clinical information, and/or additional information that would be apparent to those of skill in the art. The training data can be homogeneous or heterogeneous compared to the application data with which predictions are made. In some embodiments, a system enabled by this disclosure may be at least partially operated on local computers, distributed computing infrastructure, and/or other computer configurations. Additionally, a system enabled by this disclosure may be customized, controlled, and monitored by command-line or interactive graphical interfaces, without limitation.

A system enabled by this disclosure may advantageously leverage a feedback pathway to substantially, continually improve the predictive capabilities of the multimodal machine learning approach. For example, multiple modes of source information may be normalized, adjusted, and otherwise adapted to facilitate constructive comparison of the included data. Additionally, information relating to results, real world application, prediction efficacy, drug interactions, treatment efficacy, patient subtypes, diagnoses, risks, outcomes, and other information predicted by the system and method enabled by this disclosure may be provided to the multimodal machine learning approach. This feedback information may be used to supplement the training of the multimodal machine learning approach, adjust weights for predictions made by the multimodal machine learning approach, and/or otherwise affect the predictive outcome or improve operation of the system and method enabled by this disclosure.

Trials and experimentation relating to biomarker and subtype identification aspects will now be discussed in greater detail in the context of examples. In at least one embodiment, a system enabled by this disclosure may apply a diagnostic and therapeutic technique to improve the understandings of cancer and its related treatments. The examples given throughout this disclosure may be provided in the context of cancer research, which is one of the leading causes of death in the United States, without limitation.

Molecular and phenotypic abnormalities have been observed in cancer biospecimens. Such abnormalities may be quantified by a combination of, but not limited to, genomic, transcriptomic, proteomic, radiomic, radio genomic, spatial transcriptomics, spatial proteomic, clinical recordings, and/or other sources of information, each of which may represent a different data modality. The modalities may be analyzed for signatures, biomarkers, and/or additional detectable characteristics. Biomarkers can be used to classify the disease into subtypes, with which clinicians may estimate patient prognosis and determine courses of actions, including standard of care and clinical trial enrollment. The biomarkers may also be used to predict, for example, single drug or combination treatment benefit. Furthermore, biomarkers may be used to predict a likelihood of adverse side-effects of otherwise beneficial drugs. Biomarker-informed targeted therapy has recognized promise—for example, Lapatinib is an FDA-approved treatment for breast cancers with HER2+ signatures (reference 1). However, the response rate of many targeted treatments remains low.

Even though some teachings in the prior art have found some useful signals from two (reference 2) modalities, improving in vivo treatment targeting with a multimodal model remains a challenging problem (reference 3). The potential value for additional modalities is suggested by recent medical findings that cancer is highly correlated with, if not caused by, modalities (reference 14) omitted or deemed insignificant in vivo.

In at least some embodiments enabled by this disclosure, provided without limitation, the use of four of the aforementioned modalities already significantly improves prediction results. Examples provided by this disclosure demonstrate uses of genomic point mutation, genomic copy number mutation, transcriptomic expression, and epigenetic methylation. The biology measured by these four illustrative modalities can be measured in a more refined manner along with radio genomic, radiomic, spatial transcriptomics, proteomic, and spatial proteomic. Therefore, the method enabled by this disclosure can inherently be applied and benefited from such extra modalities for better performance.

Additionally, provided without limitation, modalities can be leveraged for diagnosis and for treatment prognosis. To further demonstrate, without limitation, that a system enabled by this disclosure can tolerate significant data heterogeneity, the example system was trained with the model of this example on molecular profiles measured from in vitro cancer cell lines and make predictions for in vivo cancer patients' molecular profiles. In vitro cancer cell lines are used as fast, low-cost, preclinical models to systematically test for potential treatment response. These cell lines are produced to mimic cancer to some degree, but they have notable differences from in vivo cancer cells (reference 3).

Such differences are significant enough that recent multimodal models from other studies trained with in vitro data have had limited success when applied to in vivo patient data reference 4). Since heterogeneity may decrease machine learning performance, a system that can cope with significant heterogeneity also advantageously performs well on more homogeneous data scenarios. Thus, the system and outcomes enabled by this disclosure may also be used in subsequent validations such as organoids (reference 12), animal models (reference 13), or clinical trials with diverse experimental methodologies.

The methodologies enabled by this disclosure will now be discussed in greater detail. As shown in FIG. 1, a schematic of analytical workflow of treatment is provided, without limitation. A training cohort, such as in vitro cell lines data, were used to build machine learning models of this example, along with transcriptomic, genomic, epigenetic featurizers. The generated model and featurizers were applied to one or more biospecimen (such as in vivo patient data) to predict subtypes, treatment response, and biomarker importance.

In this example, in vitro cell line data was sourced from the Genomics of Drug Sensitivity in Cancer (GDSC) database (reference 7). Epigenetic DNA methylation was measured in this example by IIlumina Infinium HumanMethylation450 BeadChip. Previous known ML modeling with standard processing failed to build models generalizable to in vivo data (reference 4). Here, the example system enabled by this disclosure, combined methods formed with biotechnological knowledge such as the signal profile of the beadchip to innovate an epigenetic featurizer, advantageously improves the modality by biological trend. Methylation measurements can be numerically represented by m-values or beta-values (reference 6). Most often, methylation measurement reports only beta values, each ranging from 0 (not methylated) to 1 (fully methylated). Although simple to interpret, beta values are known to suffer from heteroscedasticity and non-linearity (reference 4) in addition to measurement noise. If M-value is not available from measurement, we calculate a M value for each beta value by M=log 2(Beta/(1−Beta)) with a per-sample floating-point epsilon to prevent floating point number overflow. Each training sample now has more than tens-to-hundreds of thousands of M values. Each M value is assigned to 0, 1, or more groups, for example by gene annotation or genomic coordinate.

Within each group of M values, subgrouping is performed depending on the nature of the machine learning task. For supervised machine learning with target variables such as drug response, M values can be sub-grouped by the degree (such as positivity and negativity) of correlations to target variables. For unsupervised learning without target variables, M values can be sub-grouped by unsupervised clustering.

The numerous subgroups can then be summarized to their own features. For example, M-values can be averaged within each subgroup, or modeling methods such as Bayesian Gaussian Mixture Model applied to denoise the signal before averaging.

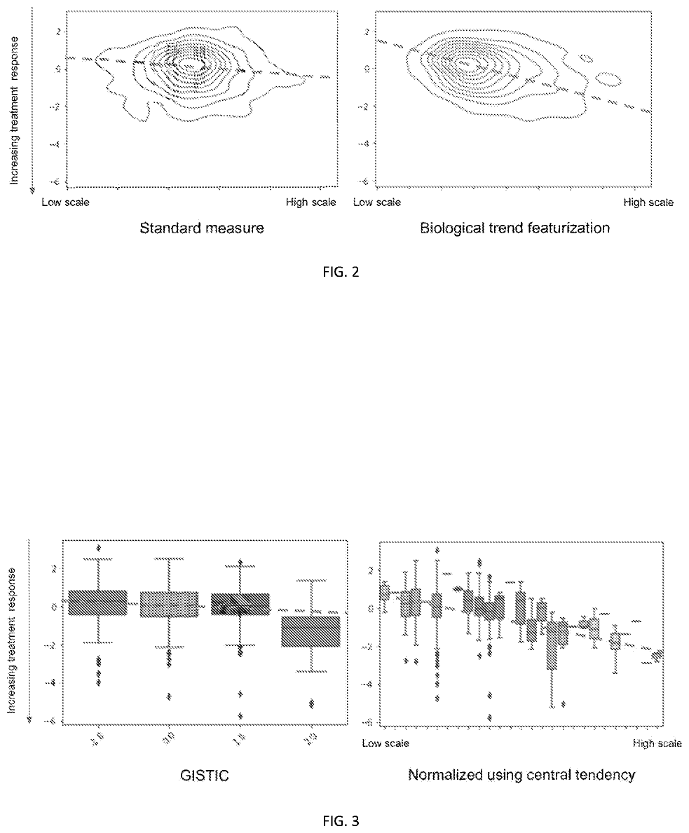

This procedure operates in a homoscedastic and a linear numerical space to denoise and enhance signal power, with results evidently demonstrated in FIG. 2 which shows the resulting feature with much higher correlation with drug response. Such denoised and consolidated feature value calculated in M-representation can also be transformed back to a denoised and consolidated beta-representation by beta=2\hat{ }M/(2\hat{ }M+1). The features can be optionally sub-selected or summarized with machine learning methods or prior knowledge, such as pathway database or significant genes from orthogonal studies of the same or different modalities.

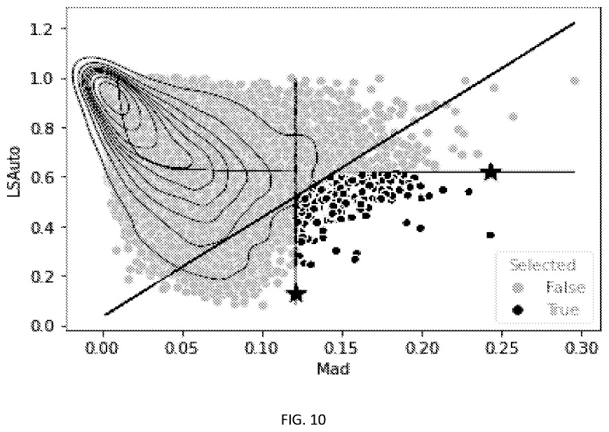

Here we further demonstrate the effectiveness of this featurization method along with a novel unsupervised machine learning featurization method on the TCGA Head and Neck Cancer cohort. Previous work used RNA measurements and methylation measurements of tumor and normal samples to identify ˜2,600 features to identify the clinically important NSD1 subtype (reference 14). Instead, we first perform the aforementioned procedure to obtain the denoised and consolidated beta-representation features. Two measures are calculated for each feature across the samples. The first measure is a measure of coherence by, for example, auto-tuned Laplacian Score (LSAuto) which estimates each feature's coherence among the samples with Laplaician score (reference 15). Importantly, LSAuto is different from the standard Laplacian score because the standard Laplacian score requires parameters set by analysts according to the nature of the data; in contrast, LSAuto automatically determines such parameters from the data in an unsupervised way. The second measure is a variance metric (such as standard deviation or mean/median absolute deviation, Mad). While the two metrics have been previously used independently for unsupervised feature selection, they were not effective for methylation data due to its noisiness and high dimensionality. We devised a novel method to select a small number of features by considering the nature of “coherence-variance” with high Mad and low LSAuto value as shown in FIG. 10.

This procedure rapidly identified, from only methylation measurement of tumor samples, 216 features. Unsupervised clustering yielded concordant results compared to the original work (TABLE 1). The table highlights Cluster 4 of current work corresponds to the NSD1-Smoking subtype in prior work. According to prior work, Cluster 4 and NSD1-Smoking should be biologically associated with mutation in the NSD1 gene, and we used an orthogonal Mutsig mutation dataset (also used by prior work) to check such association. For prior work's NSD1-Smoking subtype, 47, 28, and 5 samples have mutated, unmutated, and unknown status for NSD1 gene; thus, a known enrichment of mutation of 47/(47+28)˜62.7%. For cluster 4 of current work, 6, 17, and 5 samples have mutated, unmutated, and unknown status for NSD1 gene; thus, a known enrichment of 46/(46+17)˜73% enrichment. Therefore, clustering by this work's featurization method has a 73%-62.7%>10% improvement over previous work in terms of biological association. In summary, our featurization and machine learning method requires significantly less measurements, both 50% of measured modalities and 8% of biomarker features, yet predicts subtypes with more concordant biological properties.

TABLE 1
Cross-table showing subtype concordant between current work and previous
work. Bolded number indicates dominant correspondence between clusters
reported by current and previous work. The bolded, underlined value of
68 indicates the biologically important NSD1-Smoking subtype as pointed
out by previous work. Note that raw data from previous work pointed to
unclear separation between Non-CIMP-Atypical and Stem-like-Smoking.

| | Current Work | | | | | |
|---|---|---|---|---|---|---|
| Prior work | Cluster 1 | Cluster 2 | Cluster 3 | Cluster 4 | Cluster 5 | Cluster 6 |
| CIMP-Atypical | 28 | 84 | 1 | 0 | 1 | 0 |
| HPV+ | 3 | 0 | 59 | 0 | 0 | 2 |
| NSD1-Smoking | 4 | 0 | 0 | 68 | 6 | 2 |
| Non-CIMP-Atypical | 65 | 27 | 1 | 0 | 56 | 1 |
| Stem-like-Smoking | 6 | 4 | 2 | 0 | 48 | 60 |

In this example, genomic features were generated from two data modalities. DNA samples were measured by DNA sequencing and Affymetrix Genome-Wide Human SNP Array 6.0. Recent works (references 2, 4) that attempted to train in vitro and to predict in vivo did not report successful use of the readily available copy number modality, potentially due to batch heterogeneity such as in vivo and in vitro specimen differences (reference 3), in addition to measurement noise. Using a system and method enabled by this disclosure, the example system advantageously innovates a genomic featurizer using biotechnological knowledge, such as molecular sequence alignment tendencies (reference 8), to significantly improve the resolution and quality of the modalities while tolerating significant noise and data heterogeneity. DNA mutation calls can be selected by, for example, confidence (measurement coverage, measurement quality, mapping quality, statistical significance, and the composition of reference loci, etc.), variant effect (location, exonic/intronic, consequence and sequence ontology, SIFT, PolyPhen, etc), prior knowledge (pathway database or significant genes from orthogonal studies of the same or different modalities). The selected calls can then be featurized by, for example, absence/present for a loci/gene or a continuous value, such as 0 to 1 for allelic frequency. Even more significantly, genomic copy number aberration measurements can be heterogeneous in nature among different samples because of sample preparation, measurement technology and the nature of the samples. For example, in vivo cell lines can have overall instability in copy number change compared to that of in vitro patients. We computationally overcome these heterogeneities by computing a within-sample reference level followed by transforming the measured values with that per-sample reference. For each training/test sample, the within-sample reference level can be computed by taking, for example, the median or mean of a list of measured values of the respective sample. For each sample, the measured copy numbers can then be normalized by, for example, subtraction or division relative to the within-sample reference level. The features can be optionally sub-selected or summarized with prior knowledge, such as pathway database or significant genes from orthogonal studies of the same or different modalities. The feature improvement is evident in FIG. 3.

In an embodiment enabled by this disclosure, transcriptomic expression profiles were measured by RNA sequencing. Using biotechnological knowledge, such as molecular sequence alignment tendencies (references 8, 9), a transcriptomic featurizer was built that is applicable to both in vitro cell lines and in vivo human patients. Transcriptomic measurement quantifies the relative abundance of RNA molecules in a sample and normalization from raw count is often needed. Further, batch effects must be properly corrected such that, for example, a model trained on in measurements on vitro cell lines is applicable to in vivo patients. We perform a transferable normalization on the training set. The procedure yields a featurizer which is applicable to the training set and to any previously unknown, future test samples. We first normalize RNASeq counts per sample by, for example, transcripts per million (TPM) and log(x+1) normalization. Each sample would then have an expression value between 0 and positive infinity for each measured gene or transcript. For each sample, the expression values across genes/transcripts are sorted by ascending/descending order. Each sample would have an ascendingly/descendingly-ranked list of expression values, corresponding to potentially different order of genes/expressions. Across all training samples, for each rank (for example, lowest, second-lowest, etc), a value is computed by a summary, such as average, median. This list of summarized values, or ranked-value, is then used to featurize the training data (during training) or previously unknown test samples (during model application). For each training/test sample, the expression values are sorted to get the rank of each gene, each gene is then assigned the ranked value. Unlike commonly used rank normalization that keeps only the rank, this procedure ensures that both the numerical range and power scaling is the same between the training and test set, without other procedures such as re-calibration upon getting new samples. The features can be optionally sub-selected or summarized with prior knowledge, such as pathway database or significant genes from orthogonal studies of the same or different modalities.

From the resulting features, including candidate biomarkers, machine learning was performed on the cell line data (reference 7) to generate insights and models. The process included the use of featurizers, which prepared and optionally transformed the measurements from molecular measurement values into numerical or categorical values leveraged by ML methodologies. Examples of ML methodologies for training and prediction purposes include, without limitation, k-means clustering, hierarchical clustering, spectral clustering, gaussian mixtures, nearest neighbors, random forests, gradient boosting, or neural networks.

Treatment response models were built from data of one or more modalities. For a given training partition, model parameters were chosen by tenfold cross validation Reference 11). For a given set of features and treatments, one or more numerical or categorical variables can be predicted by the models.

The predictions created by models enabled by this disclosure may be interpreted in different ways. For instance, without limitation, a particular measure can range from negative infinity to positive infinity, with zero or above indicating that the treatment is not effective, and lower values indicating better treatment effectiveness.

From data of one or more modalities, unsupervised and/or supervised clustering was performed based on similarity, dissimilarity, and/or differential response, without limitation. The resulting clusters may be interpreted as subtypes with distinct biomarker profiles. From these identified clusters, a classifier may be used to predict the subtypes of previously unseen samples. The subtypes can be further characterized and used, without limitation, to estimate disease prognostic, select patients for clinical trials, and/or determine treatment plans.

To evaluate model performance before application to in vivo data, a tenfold cross-validation (reference 11) was performed of the above method within the cell lines. The above illustrative methodology was then applied to all cell line data in the GDSC database for final models and applied to in vivo patient data from the Cancer Genome Atlas Program (TCGA) (reference 10). The normalizer and model derived from the training step was used to predict treatment response. Patient data with DNA point mutations by sequencing, DNA copy number variations measured by the Affymetrix Genome-Wide Human SNP Array 6.0, gene expressions measured by RNA sequencing, and DNA methylations measured by Illumina Infinium HumanMethylation450 BeadChip microarray were considered for the predictive analytics.

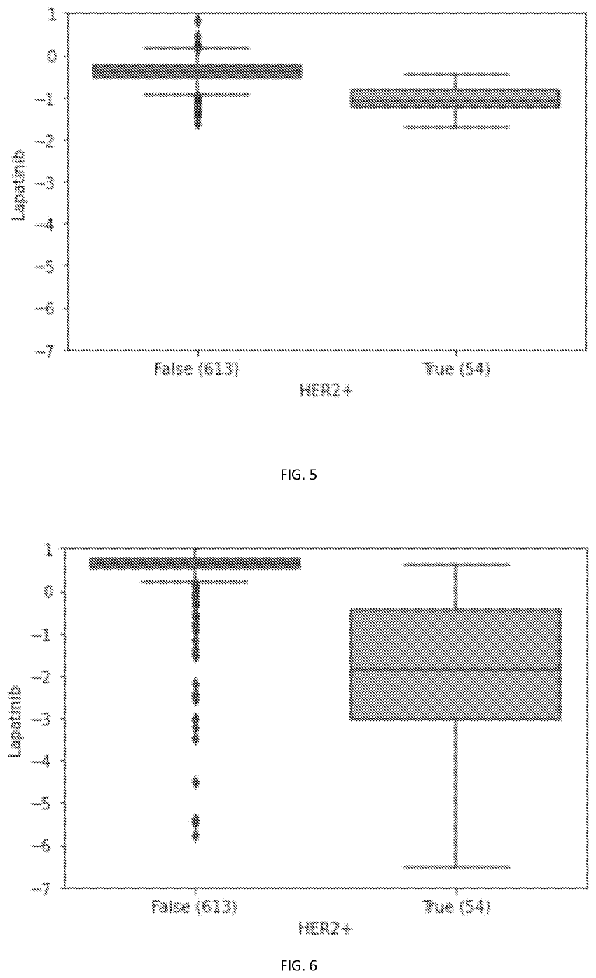

The additional diagrams will now be summarized for reference with discussion of the following examples, trials, and embodiments, without limitation. FIG. 2 provides an illustrative contour plot of treatment response versus an epigenetic feature extracted via standard measure (reference 6) (left diagram labeled “standard measure”) and a biological trend featurizer enabled by this disclosure (right diagram labeled “biological trend featurization”), without limitation. Compared to the standard method, the novel biological trend featurization method enabled by this disclosure yielded a tighter dispersion as well as higher correlation with treatment response. Many epigenetic features of the type above were compiled, along with features from other modalities during the training stage, yielding improved model performance. Further, FIG. 6 demonstrates and illustrates an embodiment in which such featurization was translatable to in vivo samples.

FIG. 3 provides an illustrative box plot of treatment response versus a genomic copy number feature extracted by the standard GISTIC method (reference 5) (left diagram labeled “GISTIC”) versus a genomic featurizer utilizing a central tendency approach enabled by this disclosure (right diagram labeled “Normalized using central tendency”). Compared to the standard method, the novel method enabled by this disclosure advantageously yielded a feature with higher resolution as well as exhibited a higher correlation with treatment response. Many genetic features of the type above were compiled, along with other modalities during the training stage, which yielded improved model performance. Further, FIG. 6 demonstrates an embodiment in which such featurization was translatable to in vivo samples.

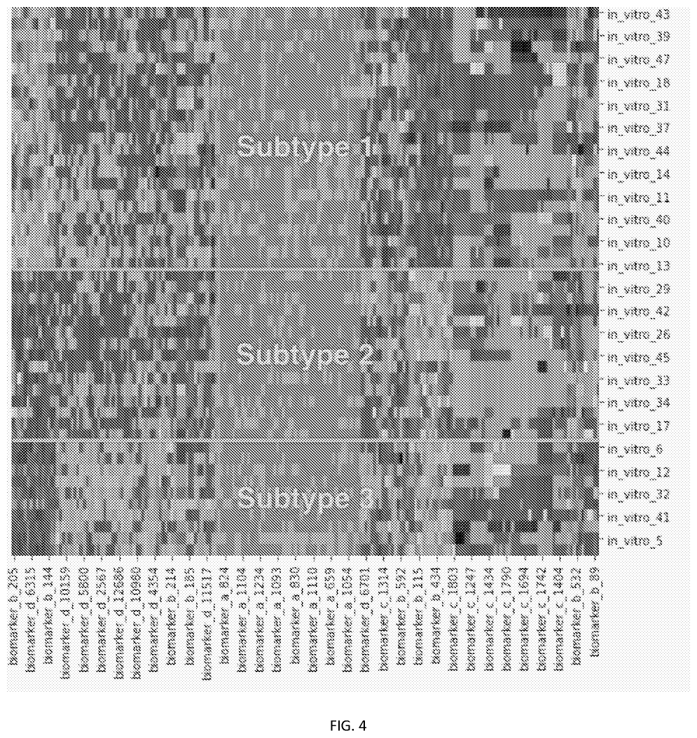

FIG. 4 provides an illustrative visualization of subtyping based on multimodal features. X-axis is a range of biomarkers from different modalities and y-axis is a range of samples, without limitation. Lighter color denotes increasing feature values. Darker color denotes decreasing feature values. Subtypes 1, 2, and 3 are grouped together. The horizontal stripe of each subtype exhibits different color trends along the biomarker X-axis.

FIG. 5 provides a box plot of Lapatinib response predictions calculated using only mutation and RNA expression modalities without limitation. Increasingly negative numbers indicate higher treatment response. HER2+ status is determined orthogonally by immunohistochemistry (IHC) and fluorescence in situ hybridization (FISH) clinical data not used by the model. The predicted value for Lapatinib sensitivity is more negative for the True group than False group, indicating that the HER2+ group is more responsive to Lapatinib treatment. However, in agreement with known research (reference 2), the False group is also predicted to be responsive (negative values).

FIG. 6 provides a box plot of Lapatinib response predictions calculated using mutation, RNA expression, DNA copy number variation, and DNA methylation modalities. Increasingly negative numbers indicate higher treatment response, without limitation. HER2+ status is determined orthogonally by IHC and FISH clinical data not used by the model. The predicted value for Lapatinib sensitivity is more negative for the True group than False group, indicating that the HER2+ group is more responsive to Lapatinib treatment. Unlike the diagram shown in FIG. 5, the False group is predicted to be less responsive (mostly positive values), in concordance with FDA guidelines but in contrast to the research compared with the chart of FIG. 5 (reference 2).

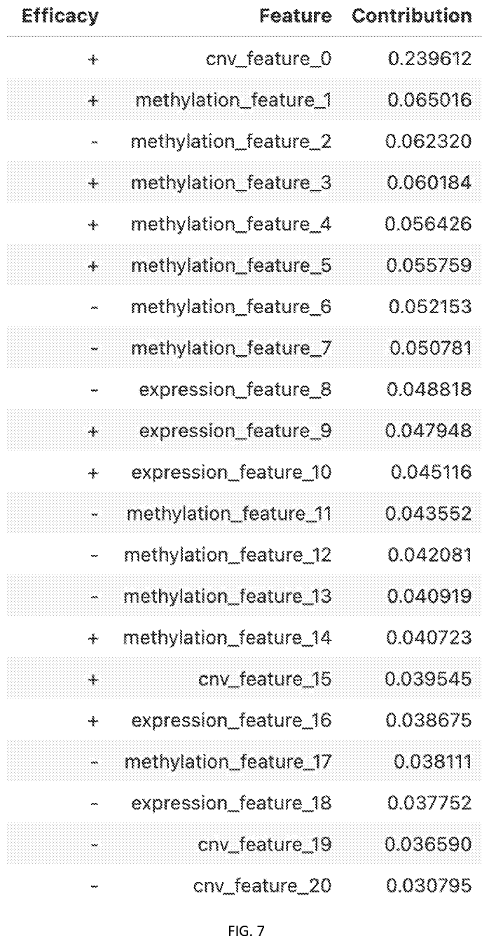

FIG. 7 provides top features (biomarker) along with their degree of efficacy for the predicted treatment response, without limitation. Constructive or destructive contribution of a biomarker is denoted as + or −, respectively. Higher contribution value means more significant effect.

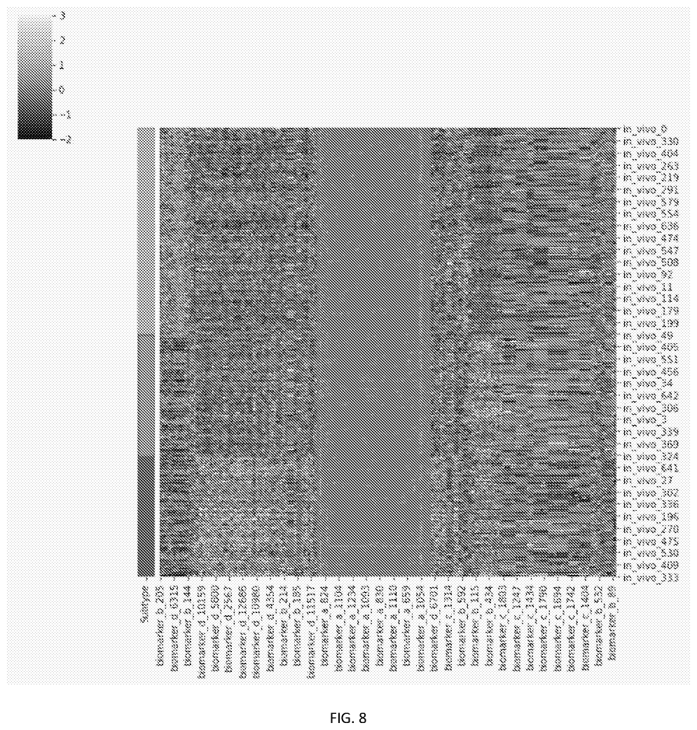

FIG. 8 provides a heatmap visualization of subtypes predicted for in vivo patients, without limitation. The Y-axis values are sorted by predicted subtype, as annotated by the 3 bars of different shades on the left for subtype 1/2/3. In the interest of clearly presenting the example, color schema is adjusted to match that as shown in FIG. 4. Lighter color denotes increasing feature values. Darker color denotes decreasing feature values. The horizontal stripe of each subtype exhibits different color trends along the biomarker X-axis.

The results and method validation aspects will now be discussed in greater detail with reference to FIGS. 1-8, without limitation. A model featurizer enabled by this disclosure significantly improved the signal-to-noise ratios of genomic and epigenetic features as seen in FIG. 2, without limitation. Additionally, FIG. 3 shows that the resolution and significance of a copy number feature is greatly enhanced by the normalization by central tendency in some embodiments. Furthermore, FIG. 2 shows that the correlation of an epigenetic feature correlation was noticeably enhanced by the biological trend featurization. FIG. 6 demonstrates an embodiment in which the results are robust even with data heterogeneity as significant as in vitro−in vivo differences.

After subtyping computation, the subtype's biological marker trend was visualized in FIG. 4, without limitation. Three subtypes were identified, and different trends were observed.

TABLE 2
Combining modalities decreases the tenfold cross-validated
root mean squared error for the Lapatinib prediction for
GDSC cell lines. Lower error means higher accuracy.

| | | Copy | | GDSC CV |
|---|---|---|---|---|
| Mutation | Expression | Number | Methylation | RMSE Error |
| ✓ | | | | 1.0240 |
| ✓ | ✓ | | | 0.9568 |
| ✓ | | ✓ | | 0.9784 |
| ✓ | | | ✓ | 0.9262 |
| ✓ | ✓ | ✓ | ✓ | 0.9217 |

Table 2 shown above demonstrates the effectiveness of combining multiple modalities according to the teachings of this disclosure. The RMSE error measure dropped from 1.0240 for a model using only DNA mutation to 0.9217 for a model following the multimodal methods enabled by this disclosure with four modalities combined.

Lapatinib has been recently approved by the U.S. Food and Drug Administration to treat breast cancers with HER2+ signatures. Experimentation using a system and method enabled by this disclosure applied the disclosed model trained on cell lines to the multimodal molecular data of the breast cancer patients in the TCGA project. The TCGA project also determined the HER2+ signature orthogonally using immunohistochemistry (IHC) and/or fluorescence in situ hybridization (FISH). FIG. 5 (mutation+expression only) and FIG. 6 (mutation+expression+copy number+methylation) demonstrate embodiments in which the treatment response predicted by the model enabled by this disclosure, with increasingly negative numbers indicating higher treatment effectiveness. As a validation, the model prediction (using only mutation and expression modalities in FIG. 5) agreed with the previous findings (reference 2).

The difference from adding extra modalities was significant and advantageous. First, the model, trained with cell lines and without any IHC and FISH HER2+ data correctly predicted differential treatment response in concordance with the FDA's guidelines. Second, the inclusion of copy number and methylation modalities resulted in a significantly more pronounced spread between the HER2+ and non-HER2+ response to Lapatinib. For example, the upper quantile of the HER2+ group was well below the lower whisker of the False group. This means that patients' potential response to treatment can be differentiated more accurately.

Last and most importantly, the model without copy number and methylation predicts Lapatinib to be effective (negative number prediction) on most patients regardless of HER2+ group membership, while the model trained on all four of mutation, expression, copy number, and methylation predicted mostly positive numbers for the nonHER2+ group, indicating that the majority of patients without HER2+ signatures would not benefit from Lapatinib treatment. The model recommending Lapatinib primarily for HER2+ positive patients in concordance with the FDA approval offered evidence for the validity of the method. Further, the table illustrated in FIG. 7 shows an embodiment in which the model's ability to report the treatment efficacy attributed to different biomarkers.

Subtype predictions were made for each in vivo patient. Each subtype was grouped together and plotted against the biomarker values in FIG. 8, which shows that the biomarker trend is different between the subtypes, without limitation. Remarkably, even under significant heterogeneity between cell lines and in vivo, featurizers and models enabled by this disclosure combined to learn generalizable innate subtypes from the cell lines and to classify in vivo patients, with distinct biomarker trends among the subtypes.

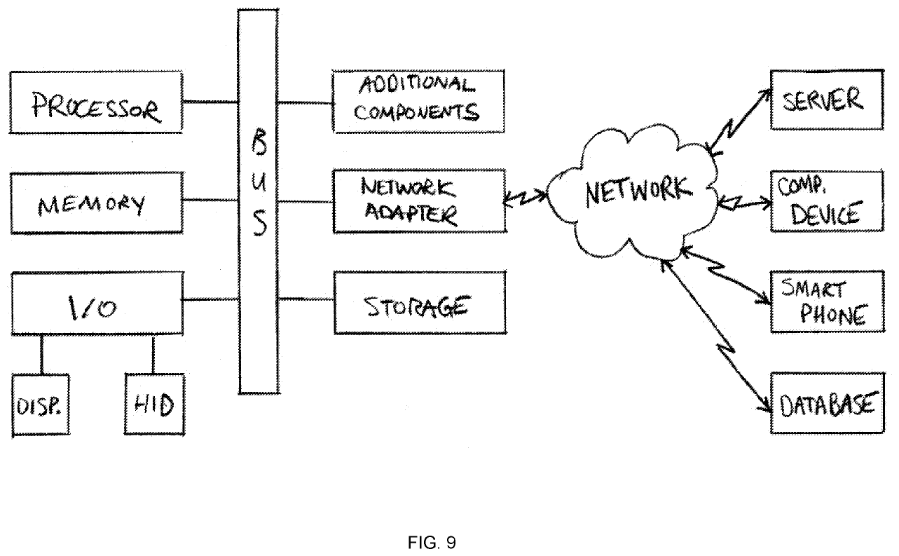

Referring now to FIG. 9, an illustrative computerized device will now be discussed in greater detail, without limitation. The computerized device may include a processor, memory, network controller, and optionally an input/output (I/O) controller. Skilled artisans will appreciate additional embodiments of a computerized device that may omit one or more of the aforementioned components or include additional components without limitation. The processor may receive and analyze data. The memory may store data, which may be used by the processor to perform the analysis. The memory may also receive data indicative of results from the analysis of data by the processor.

The memory may include volatile memory modules, such as random access memory (RAM), or non-volatile memory modules, such as flash-based memory. Skilled artisans will appreciate the memory to additionally include storage devices, such as, for example, mechanical hard drives, solid state data, and removable storage devices.

The computerized device may also include a network controller. The network controller may receive data from other components of the computerized device to be communicated with other computerized devices via a network. The communication of data may be performed wirelessly. More specifically, without limitation, the network controller may communicate and relay information from one or more components of the computerized device, or other devices and/or components connected to the computerized device, to additional connected devices. Connected devices are intended to include data servers, additional computerized devices, mobile computing devices, smart phones, tablet computers, and other electronic devices that may communicate digitally with another device. In one example, the computerized device may be used as a server to analyze and communicate data between connected devices.

The computerized device may also include an I/O interface. The I/O interface may be used to transmit data between the computerized device and extended devices. Examples of extended devices may include, but should not be limited to, a display, external storage device, human interface device, printer, sound controller, or other components that would be apparent to a person of skill in the art. Additionally, one or more of the components of the computerized device may be communicatively connected to the other components via the I/O interface.

The components of the computerized device may interact with one another via a bus. Those of skill in the art will appreciate various forms of a bus that may be used to transmit data between one or more components of an electronic device, which are intended to be included within the scope of this disclosure.

The computerized device may communicate with one or more connected devices via a network. The computerized device may communicate over the network by using its network controller. More specifically, the network controller of the computerized device may communicate with the network controllers of the connected devices. The network may be, for example, the internet. As another example, the network may be a WLAN. However, skilled artisans will appreciate additional networks to be included within the scope of this disclosure, such as intranets, local area networks, wide area networks, peer-to-peer networks, and various other network formats. Additionally, the computerized device and/or connected devices may communicate over the network via a wired, wireless, or other connection, without limitation.

In operation, a method may be provided for using machine learning to predict medical diagnostic and/or therapeutic methods using multimodal data. For example, illustrative diagnostic methods may be associated with biomarkers and subtypes, without limitation. Those of skill in the art will appreciate that the disclosed methods are provided to illustrate an embodiment of the disclosure and should not be viewed as limiting the disclosure to only those methods or aspects. Skilled artisans will appreciate additional methods within the scope and spirit of the disclosure for performing the operations provided by the examples given throughout this disclosure after having the benefit of this disclosure. Such additional methods are intended to be included by this disclosure.

The aforementioned training and prediction analytics can be executed on a local computer, single computer, multiple computers, network-connected computer, distributed computing infrastructure, and/or other computing platforms that will be appreciated by those of skill in the art after having the benefit of this disclosure. For example, a system and method enabled by this disclosure can be operated on a cloud computing platform.

The operation can be customized, controlled, and monitored by a text terminal, graphical user interface, or other computer interface. For example, a graphical interface can be a structured form, an interactive drawing form, or other form as will be appreciated by skilled artisans with which a user may interact. For long-running tasks, the status of the processing for any input data set may be displayed and/or monitored by a user. Additionally, a user may opt to be proactively notified when the processing has completed. The results, including, but not limited to, drug response predictions, will be available at a location or address selected or specified by the user.

While various aspects have been described in the above disclosure, the description of this disclosure is intended to illustrate and not limit the scope of the invention. The invention is defined by the scope of the claims of a corresponding nonprovisional utility patent application and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the claims of a corresponding nonprovisional utility patent application.

### REFERENCES

(REFERENCE 1) Ryan Q, Ibrahim A, Cohen M H, Johnson J, Ko C W, Sridhara R, Justice R, Pazdur R. FDA drug approval summary: lapatinib in combination with capecitabine for previously treated metastatic breast cancer that overexpresses HER-2. Oncologist. 2008 October; 13(10):1114-9. doi: 10.1634/theoncologist.2008-0816. Epub 2008 Oct. 10. PMID: 18849320.
(REFERENCE 2) Rydzewski N R, Peterson E, Lang J M, et al. Predicting cancer drug TARGETS—Treatment Response Generalized Elastic-net Signatures. NPJ Genom Med. 2021; 6(1):76. Published 2021 Sep. 21. doi:10.1038/s41525-021-00239-z
(REFERENCE 3) Jean-Pierre Gillet, Sudhir Varma, Michael M. Gottesman, The Clinical Relevance of Cancer Cell Lines, JNCI: Journal of the National Cancer Institute, Volume 105, Issue 7, 3 Apr. 2013, Pages 452-458, https://doi.org/10.1093/jnci/djt007
(REFERENCE 4) Miranda S P, Baião FA, Fleck J L, Piccolo S R (2021) Predicting drug sensitivity of cancer cells based on DNA methylation levels. PLoS ONE 16(9): e0238757. https://doi.org/10.1371/journal.pone.0238757
(REFERENCE 5) Mermel, C. H., Schumacher, S. E., Hill, B. et al. GISTIC2.0 facilitates sensitive and confident localization of the targets of focal somatic copy-number alteration in human cancers. Genome Biol 12, R41 (2011). https://doi.org/10.1186/gb-2011-12-4r41
(REFERENCE 6) Du, P., Zhang, X., Huang, C C. et al. Comparison of Beta-value and Mvalue methods for quantifying methylation levels by microarray analysis. BMC Bioinformatics 11, 587 (2010). https://doi.org/10.1186/1471-2105-11-587
(REFERENCE 7) Yang W, et al. Genomics of drug sensitivity in cancer (GDSC): a resource for therapeutic biomarker discovery in cancer cells. Nucleic Acids Res. 2013; 41:D955-D961. doi: 10.1093/nar/gks1111
(REFERENCE 8) Hassanpour, S H, et al, Review of cancer from perspective of molecular. Journal of Cancer Research and Practice 4 4 (2017) and references therein.
(REFERENCE 9) Stark, R., Grzelak, M., & Hadfield, J. (2019). RNA sequencing: the teenage years. Nature Reviews Genetics, 20(11), 631-656.
(REFERENCE 10) The Cancer Genome Atlas Research Network., Weinstein, J., Collisson, E. et al. The Cancer Genome Atlas Pan-Cancer analysis project. Nat Genet 45, 1113-1120 (2013). https://doi.org/10.1038/ng.2764
(REFERENCE 11) Hastie T, R Tibshirani, Friedman J, The Elements of Statistical Learning, Springer (2009)
(REFERENCE 12) Kim, J., Koo, BK. & Knoblich, J. A. Human organoids: model systems for human biology and medicine. Nat Rev Mol Cell Biol 21, 571-584 (2020). https://doi.org/10.1038/s41580-020-0259-3
(REFERENCE 13) de Ruiter J. R., Wessels L. F. A., Jonkers J. 2018 Mouse models in the era of large human tumour sequencing studies Open Biol.
(REFERENCE 14) NSD1 inactivation defines an immune cold, DNA hypomethylated subtype in squamous cell carcinoma, K Brennan, et al. Sci Rep. 2017; 7: 17064
(REFERENCE 15) Laplacian Score for feature selection, X. He et. Al. Advances in Neural Information Processing Systems 18 (NIPS 2005)

1. An apparatus for computational machine learning-based normalization, harmonization, and improvement of data signals from one or more modalities of measurements, comprising:
a. a data input module configured to receive data from one or more biospecimens with homogeneous or heterogeneous natures, including but not limited to genomic, transcriptomic, proteomic, and epigenetic data;
b. a preprocessing module configured to execute M-space signal partition, summary and smoothing on methylation signal data;
c. a preprocessing module configured to execute transferable quantile normalization for transcriptomic signal data;
d. a preprocessing module configured to execute reference-free DNA copy number estimation module adapted to estimate DNA copy numbers from heterogeneous sample types and measurement methods;
e. a feature selection module configured to execute Coherence-Variance unsupervised feature selection or target-aware clustering for supervised feature summary; and/or
f. a machine learning module incorporating supervised and/or unsupervised learning methods to process said data, said machine learning module further configured to perform patient stratification, biomarker discovery, and prediction of drug response based on said data.

2. The apparatus of claim 1, wherein the preprocessing module further comprises means to execute M-space signal partition, summary and smoothing by applying an algorithm for the enhancement of methylation signal data.

3. The apparatus of claim 1, wherein the preprocessing module further comprises means to execute Coherence-Variance unsupervised feature selection or target-aware clustering for supervised feature summary by employing steps for identifying relevant features from said data.

4. The apparatus of claim 1, wherein the preprocessing module further comprises means to execute transferable quantile normalization for transcriptomic signal data using steps to achieve harmonization and data accuracy improvement.

5. The apparatus of claim 1, wherein the reference-free DNA copy number estimation module employs steps to estimate DNA copy numbers from biospecimens with highly heterogeneous natures and varying measurement methods, eliminating the need for reference samples.

6. A method for improving the accuracy of biomarker discovery, patient stratification, and prediction of drug response, comprising the combination of 2 or more of following steps:
a. receiving data from one or more modality measurements of one or more biospecimens;
b. applying M-space signal smoothing to methylation signal data;
c. performing transferable quantile normalization for transcriptomic signal data;
d. estimating DNA copy numbers using a reference-free method for heterogeneous sample types and measurement methods;
e. executing Coherence-Variance unsupervised feature selection or target-aware clustering for supervised feature summary; and
f. utilizing supervised and/or unsupervised learning methods to process said data for biomarker discovery, patient stratification, and prediction of drug response.

7. A method for computational machine learning-based normalization, harmonization, and improvement of data signals from one or more modalities of measurements, comprising:
a. receiving data using a data input module from one or more biospecimens with homogeneous or heterogeneous natures, including but not limited to genomic, transcriptomic, proteomic, and epigenetic data;
b. executing using a preprocessing module M-space signal partition, summary and smoothing on methylation signal data, and
c. transferable quantile normalization for transcriptomic signal data;
d. estimating using a reference-free DNA copy number estimation module adapted to estimate DNA copy numbers from heterogeneous sample types and measurement methods; and
e. performing Coherence-Variance unsupervised feature selection or target-aware clustering for supervised feature summary
f. incorporating supervised and/or unsupervised learning methods to process said data using a machine learning module, said machine learning module further configured to perform patient stratification, biomarker discovery, and prediction of drug response based on said data.

8. A method of generating a therapeutic treatment response prediction, a biomarker prediction and a patient subtype prediction for a patient using at least one biospecimen from the patient, the method comprising:
a. training a machine learning module with modality data, the modality data comprising data from genomics, transcriptomics, proteomics, radiomics, radio genomics, spatial transcriptomics, spatial proteomics, and/or other clinical information from multiple patients, wherein the machine learning module analyzes the modality data, and wherein the analysis of the modality data comprises the identification and ranking of transcriptomic, genomic and epigenetic biomarkers of the modality data;
b. generating a model from step a. and data from the at least one biospecimen from the patient, the data from the patient comprising genomic, transcriptomic, proteomic, radiomic, radio genomic, spatial transcriptomic, spatial proteomic, and/or other clinical information; and
c. generating from the model from step b., a treatment response prediction, a biomarker prediction, and a patient subtype prediction for the patient.

9. An apparatus for predicting treatment responses, biomarkers, and patient subtypes concerning a therapeutic treatment for patients, the apparatus comprising:
a. a machine learning module that accepts modality data from patients for analysis, the modality data comprising data from genomics, transcriptomics, proteomics, radiomics, radio genomics, spatial transcriptomics, spatial proteomics, and/or other clinical information from multiple patients, and the analysis comprises identifying and ranking transcriptomic, genomic and epigenetic biomarkers of the modality data; and
b. a model generating module that accepts the analysis from the machine learning module and data from at least one biospecimen from a patient, the data from the patient comprising genomic, transcriptomic, proteomic, radiomic, radio genomic, spatial transcriptomic, spatial proteomic, and/or other clinical information, and wherein the model generating module generates a model that predicts treatment response, biomarkers, and patient subtypes with respect to treatments for that patient.
